(19) United States
(12) Patent Application Publication
(10) Pub. No.: US 2016/0257490 A1
(43) Pub. Date: Sep. 8, 2016

(54) DISPOSABLE COMPOST BIN

(71) Applicant: Platform, Inc., Seattle, WA (US)

(72) Inventors: Glenn M. Geisendorfer, Seattle, WA (US); Gabriel I. Goldman, Seattle, WA (US)

(73) Assignee: Platform, Inc., Seattle, WA (US)

(21) Appl. No.: 15/056,568

(22) Filed: Feb. 29, 2016

Related U.S. Application Data
(60) Provisional application No. 62/128,467, filed on Mar. 4, 2015.

(51) Int. Cl. B65F 1/14; C12M 3/00; C12M 1/00

(52) U.S. Cl. CPC B65F 1/1615; B65F 1/141; C12M 23/02; C12M 23/46; B65F 2001/1489; B65F 2250/105

(57) ABSTRACT

A disposable compost bin for receiving compostable materials, the bin comprised of (i) a container made from a compostable material, the container having a continuous inner surface, a closed bottom portion, a continuous wall defining an open upper portion, and a continuous lid receiving edge on the open upper portion, and (ii) a removable lid having a substantially rectangular shape and a continuous outer edge, the lid insertably placed on the continuous lid receiving edge of the container for sealing the received compostable materials, the removable lid forming a vacuum-like seal when insertably placed on the continuous lid receiving edge.

### RELATED APPLICATIONS

The present application is based upon and claims the benefit of priority from Provisional Application No. 62/128,467 filed on Mar. 4, 2015, the contents of which are hereby incorporated by reference.

### FIELD

The present disclosure relates generally to food waste management, and in particular but not exclusively, relates to an apparatus and method for composting food waste using a disposable compost bin.

### BACKGROUND

There is a growing interest among consumers and businesses in identifying and pursuing sustainable methods for recycling resources and for using existing natural resources more efficiently to reduce the deleterious effect of human exploitation of such resources on this planet. As a direct result of such heightened environmental consciousness, a significant grassroots movement has gained ground across the world that actively promotes natural alternatives to the heavily process-oriented industrialized options that have been used up to the present time. In particular, there is growing interest among consumers, businesses and local governments in more effective and efficient uses of organic matter such as food waste. In lieu of routine disposal of such organic matter with conventional inorganic trash, a growing movement in many communities is proving that composting is a viable natural alternative for achieving greater efficiencies and uses of organic matter. Local governments are also beginning to mandate that such organic matter be disposed of in compost heaps instead of added to the already burdensome collections of trash and other refuse in conventional trash dump sites.

Composting by its very nature involves the compilation of organic matter in heaps that facilitate the natural decomposition of the matter into raw materials that can improve soil structure, increase nutrient content, reduce water consumption, and control plant diseases and insects than might otherwise be present in sterile soil environments. Despite its many advantages, effective composting of discarded food matter requires the use of composting accessories such as bags, liners and filters and generally can result in an untidy mess that often requires the frequent replacement of these accessories. Since effective composting requires constant attention to the turning and tilling of soil, the use of these accessories often complicates the efforts of those who attempt to systemically compost their food waste.

In addition to the complexities associated with composting using available accessories, optimal composting requires constant maintenance and attention to soil augmentation and soil aeration. Ineffective composting results in uneven decomposition across a compost heap that can ultimately lengthen the time needed to achieve the desired benefits of composting. Efficient decomposition of organic matter in a compost heap can be achieved if aeration (i.e., a considerable amount of oxygen), moisture, particle size, and sufficient sources of carbon and nitrogen are present. Absent effective aeration, anaerobic decomposition occurs resulting in slower decomposition that can in turn lead to the production of unpleasant odors from the putrefactive breakdown of organic materials. Thus, it is preferable to achieve the results of composting on an aerobic basis, particularly if composting is to be accomplished in high-density locations, such as on the rooftops of apartment complexes or condominium buildings in major metropolitan centers. However, aerobic composting alone is not sufficient and a need exists for an apparatus that can accelerate and improve the composting process while reducing the need for additional maintenance and the bags, liners and filters referred to earlier.

Despite the recognized positive benefits of composting, for people who live in high-density metropolitan areas relatively few options or opportunities exist for effective composting. In addition, increased living density often means less living space in living units such as apartment units, condominium units and other cooperative living arrangements. In such living units, there is little to no room on kitchen countertops, floors, or in other in-home locations where yet another container or object can be placed to set aside organic matter that could be discarded onto a compost heap. This is often the case even though there is a growing realization of the value of composting in urban environments and an increasing number of local and municipal jurisdictions that are requiring the segregation of organic matter from other forms of refuse and the composting of such organic matter.

Thus, there is a significant and rapidly growing need for a disposable compost bin comprised of compostable materials as well as a method for using such a bin that can provide a simplified approach for composting discarded food waste and other organic matter while also enabling, accelerating and improving aerobic composting processes with no additional maintenance or accessories. The unmet need for such a solution is of growing importance to those who live in high-density metropolitan areas where urban composting is becoming an absolute necessity since an increasing number of local and municipal jurisdictions are mandating the adoption of a solution. The need for a disposable compost bin takes on even greater importance in view of the increasing number of living units, particularly in metropolitan areas, that have less space on kitchen countertops, floors or in other locations for an effective, affordable, simple, and environmentally sustainable solution for the disposal of compostable organic matter.

### BRIEF DESCRIPTION OF THE DRAWINGS

Non-limiting and non-exhaustive embodiments are described with reference to the following figures, wherein like reference numerals refer to like parts throughout the various views unless otherwise specified.

FIG. 1 is a perspective view of a container for a disposable compost bin in an embodiment.

FIG. 2 is a perspective view of a disposable compost bin in an embodiment.

FIG. 3 is a perspective view of a disposable compost bin in an embodiment.

FIG. 4 is a top view of a disposable compost bin in an embodiment.

FIG. 5 is a frontal view of a disposable compost bin in an embodiment.

FIG. 6 is a side view of a disposable compost bin in an embodiment.

FIG. 7 is a top view of a disposable compost bin in an embodiment.

FIG. 8A is a perspective view of a disposable compost bin and a mounting means in an embodiment.

FIG. 8B is a perspective view of a disposable compost bin mounted on a side wall structure in an embodiment.

FIG. 9 is a frontal view of a disposable compost bin and a mounting means in an embodiment.

FIG. 10 is a side view of a disposable compost bin and a mounting means in an embodiment.

FIG. 11 is a top view of a mounting means for a disposable compost bin in an embodiment.

FIG. 12 is a perspective view of a mounting means for a disposable compost bin in an embodiment.

FIG. 13 is a frontal view of a mounting means for a disposable compost bin in an embodiment.

FIG. 14 is a side view of a mounting means for a disposable compost bin in an embodiment.

FIG. 15A is an illustrative view of compost bins in a composting collection resource in an embodiment.

FIG. 15B is a bottom-up view of adjacent compost bins in an embodiment.

FIG. 15C is an illustrative view of air flow between stacked compost bins in an embodiment.

FIG. 16 is a flowchart illustrating a method of using a disposable compost bin in an embodiment.

### DETAILED DESCRIPTION

In the description to follow, various aspects of embodiments of disposable compost bins will be described, and specific configurations will be set forth. Numerous and specific details are given to provide an understanding of these embodiments. The aspects disclosed herein can be practiced without one or more of the specific details, or with other methods, components, systems, or services. In other instances, structures or functions are not shown or described in detail to avoid obscuring relevant inventive aspects.

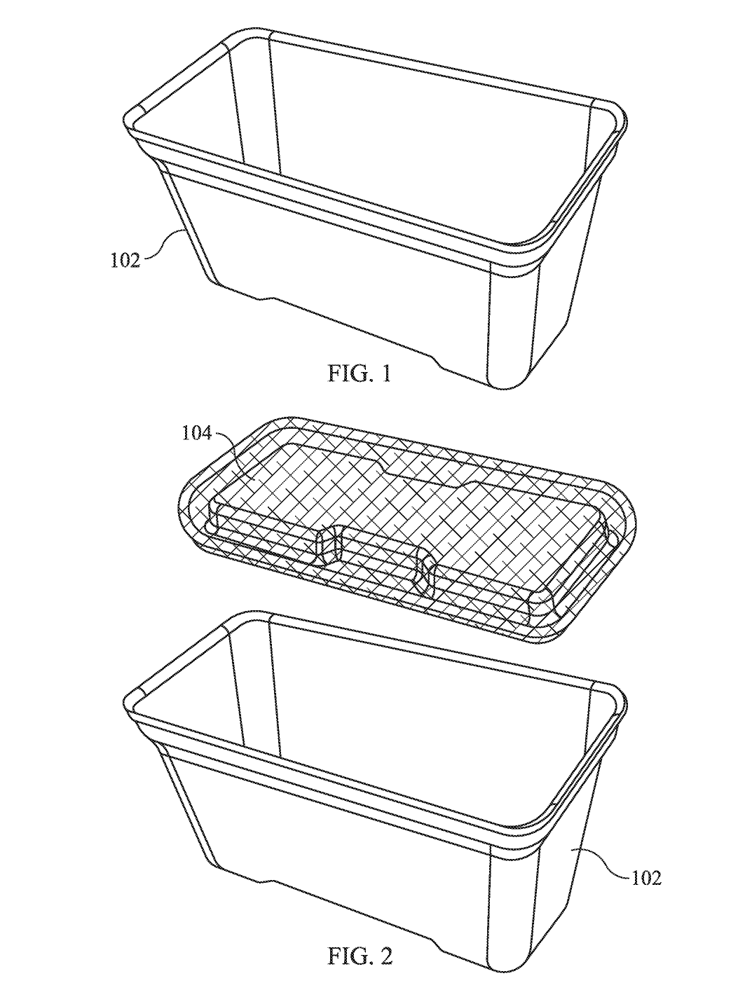

FIG. 1 illustrates an embodiment of a container for a disposable compost bin. The compost bin is comprised of a container 102 and a removable lid 104. The container 102 is comprised of compostable plant-based fiber materials and includes a continuous wall that defines an open upper portion for receiving compostable materials. The container includes a closed bottom portion 108 and includes on the upper portion a continuous lid receiving edge for receiving the removable lid 104. The container 102 in a first embodiment has a trapezoidal design with an upper portion that is off-angle from a true horizontal direction. In an alternative embodiment, the container 102 has a rectangular design with an upper portion that is likewise off-angle from the horizontal direction.

FIG. 2 illustrates a disposable compost bin comprised of a container 102 and a removable lid 104 in an embodiment. The removable lid 104 is comprised of a compostable material and has a substantially rectangular shape and a continuous outer edge. The removable lid can be inserted into a continuous lid receiving edge on an open upper portion of the container 102 for sealing received compostable materials. In forming a seal, the removable lid 104 forms a vacuum-like seal when insertably placed onto the continuous lid receiving edge. In one embodiment, both the container 102 and the removable lid 104 are made from compostable plant-based fiber materials such as wheat straw, wood fiber, sugarcane fiber or other suitable materials that are non-toxic, break down easily, and help support healthy plant growth.

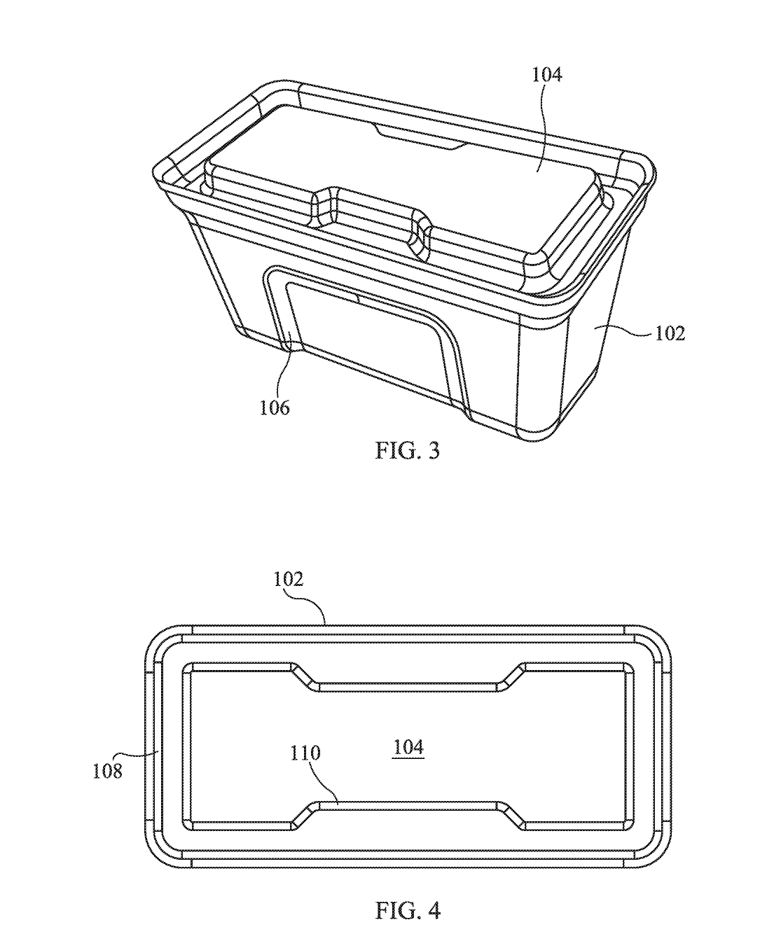

FIG. 3 is a perspective view of a disposable compost bin in an embodiment. In the illustrated embodiment, a container 102 is shown with an inserted removable lid 104 and a shaped groove 106 on the outer surface of a wall of the container 102. The shaped groove 106 is formed to receive a mounting means for the compost bin to enable the bin to be held in place in a convenient location such as the inside of a cabinet door. By placing the bin in such a location, users to will be able to minimize or eliminate prolonged contact with waste food matter after its initial disposal.

FIG. 4 is a top view showing a removable lid 104 mounted onto a container 102 of a compost bin in an embodiment. The removable lid 104 in the present embodiment has a rectangular design with an inner shaped region 110 and a continuous outer edge 108 that is insertably placed into a continuous lid receiving edge in the container 102. Although depicted as being generally lying in a horizontal plane, the top of the container is off-angle from a true horizontal direction. Likewise, the removable lid 104 inserted into the container 102 on its continuous lid receiving edge also lies in a direction on the open upper portion of the container 102 that is off-angle from the horizontal direction. Although the container 102 and the removable lid 104 can have varying dimensions, in the illustrated embodiment the outer-end to outer-end height of the container 102 is generally about 140 mm and the outer-end to outer-end width is generally about 310 mm.

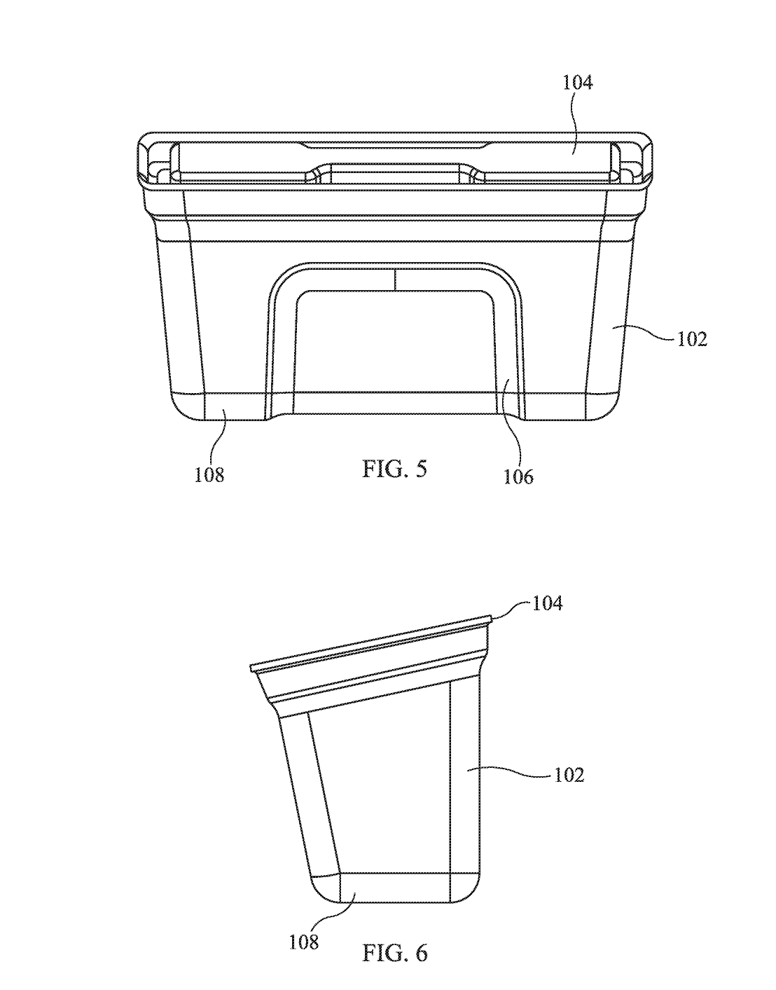

FIG. 5 is a side view of a disposable compost bin in an embodiment. In the illustrated embodiment, the compost bin includes at least one shaped groove 106, a container 102, a closed bottom portion 108 of the container 102, and a removable lid 104. As depicted in FIGS. 1-2 and here in FIG. 5, the removable lid 104, the container 102 and its closed bottom portion 108 are used to collect unwanted food waste or organic refuse and to promote aerobic composting of such materials. In alternative embodiments of the compost bin shown in FIGS. 2 and 5, the container 102 can be provided with soil amendments to accelerate and improve the internal composting process. Among the various forms of soil amendments that can be included are coffee grounds, bone meal, blood meal, vermiculite and pumice. Such soil amendments are added to the food waste and/or organic matter included in the disposable compost bin in one embodiment. In an alternative embodiment, one or more of the soil amendments are integrated into the compostable materials used to create the container 102 and the removable lid 104. In addition, the container 102 is lined with a vapor barrier for moisture retention and odor prevention, both of which are important considerations when using the compost bin in a modern urban environment such as high density apartment buildings and condominium complexes. In an embodiment, the vapor barrier is an emulsified wax additive such as Alkyl Ketene Dimer (referred to generally as “AKD”) or other compounds having similar chemical properties.

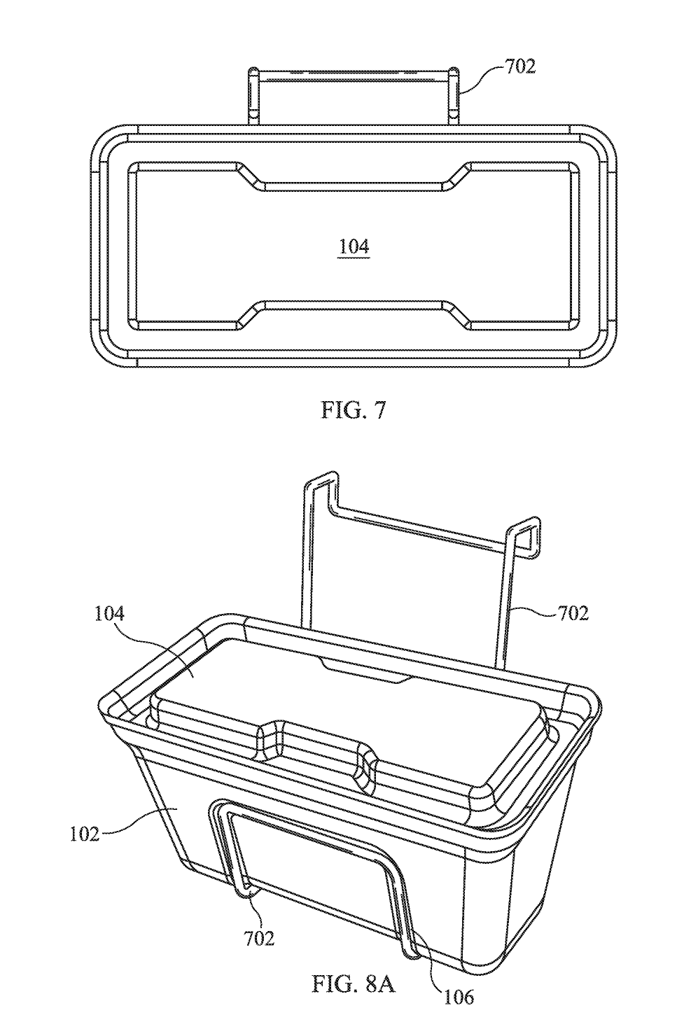

FIG. 6 is a side view of a disposable compost bin in an embodiment. As illustrated, the container 102 of the compost bin has a generally trapezoidal shape. In an alternative embodiment, the container 102 is generally rectangular in shape. The removable lid 104 is shown inserted in the container 102 at an angle from a horizontal plane in a sealed fashion after receipt and storage of compostable materials. In the illustrated embodiment and in the embodiment shown in FIG. 1, the depth of the compost bin from its highest point at the rear of the removable lid 104 when inserted into the container 102 to the lowest point of the bin on the outer-end of the bottom closed portion 108 is generally in the range of 165 to 170 mm.

FIG. 7 is a top view of a disposable compost bin with a removable lid 104 and a mounting means in an embodiment. In the illustrated embodiment, the mounting means is a metal wire-frame hanger 702. The outer end of the metal frame 702 is slidably inserted into the shaped groove 106 on the outer wall of the container 102 of the compost bin. The supporting portion of the metal hanger 702 is shown parallel to the inner wall of the container 102 in this embodiment. The metal hanger 702 enables the mounting of the disposable compost bin onto a side wall structure such as a cabinet door under a sink in a home dwelling (e.g., house, apartment, condominium, etc.). In the illustrated embodiment, the width of the container 102 from outer-end to outer-end is generally in the range of 305 to 310 mm and the length from the outer front wall of the container 102 to the rear end of the wire-frame hanger 702 is generally in the range from 175 to 180 mm.

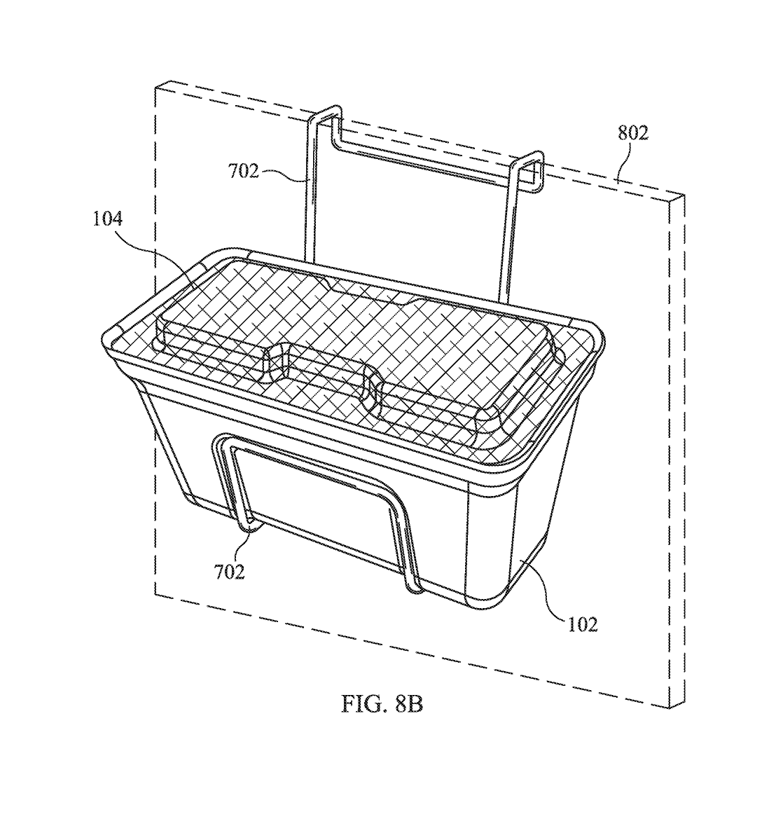

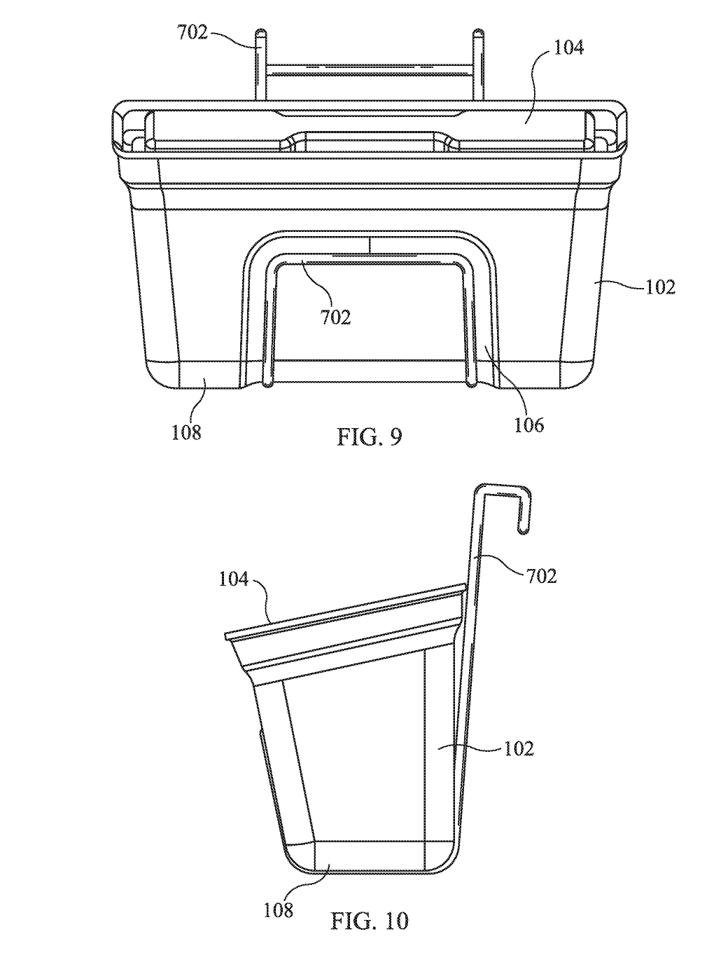

FIG. 8A is a perspective view of a disposable compost bin. As depicted, the metal wire-frame hanger 702 is used as a mounting means for the compost bin. Although the mounting means in the illustrated embodiment is a wire-frame hanger 702, in alternative embodiments the mounting means is an integrated solid-sheet metal hanger having the same general shape as the illustrated metal wire-frame hanger 702. The upper rear portion of the mounting means includes a hooked region for use in mounting the hanger 702 onto a side wall structure or other receiving structure. With the addition of the hanger 702, the disposable compost bin can be removed from a countertop and placed under a sink in a kitchen or in a different location where food waste can be discarded to avoid unpleasant smells or odors while maximizing the utility of available space used in a kitchen or other household environment. In this embodiment, the disposable bin includes a removable lid 104 mounted in the container 102 with the outer-end of the hanger 702 received within a shaped groove 106 on the outer wall of the container 102.

FIG. 8B illustrates an embodiment of a disposable compost bin mounted on a side wall structure 802. As illustrated, the metal wire-frame hanger 702 is used as a mounting means for securing a compost bin comprised of a container 102 and a removable lid 104. The side wall structure 802 in one embodiment is the inner side of a kitchen cabinet door. In alternative embodiments, the side wall structure 802 is the inner side of a pantry or storage closest or other convenient and generally accessible location for the storage of used food goods.

FIG. 9 is a side view of an embodiment of a disposable compost bin. In the illustrated embodiment, the disposable compost bin includes a metal wire-frame hanger 702 as a mounting means received within a shaped groove 106 on an outer wall of a container 102. The container 102 in this embodiment includes at least one shaped groove 106, one on an outer wall and another on the opposing outer wall. A removable lid 104 is also provided that is insertably placed into an open upper portion of the container 102 along a continuous lid receiving edge. The bin is sealed on the upper end by the removable lid 104 and on the lower end with a continuous closed bottom portion 108 of the container 102. In the illustrated embodiment, the height of the compost bin in the present embodiment from the upper side of the hanger 702 to the outer-end of the closed bottom portion is generally in the range of 210 to 215 mm. The width of the compost bin from the leftmost outer-end of the container 102 to the rightmost outer-end of the container 102 is generally in the range of 300 to 310 mm.

FIG. 10 is a side view of a disposable compost bin with a metal wire-frame hanger 702 used as a mounting means in an embodiment. As illustrated, the compost bin is comprised of a removable lid 104 insertably placed into a container 102 with a closed bottom portion 108. In the present embodiment, the height of the combined product from the highest point of the wire-frame hanger 702 to the outer bottom end of the wire-frame hanger 702 is generally in the range of 210 to 215 mm. The width of the combined product from the front edge of the removable lid 104 to the rear edge of the wire-frame hanger 702 is generally in the range of 170 to 180 mm in the illustrated embodiment.

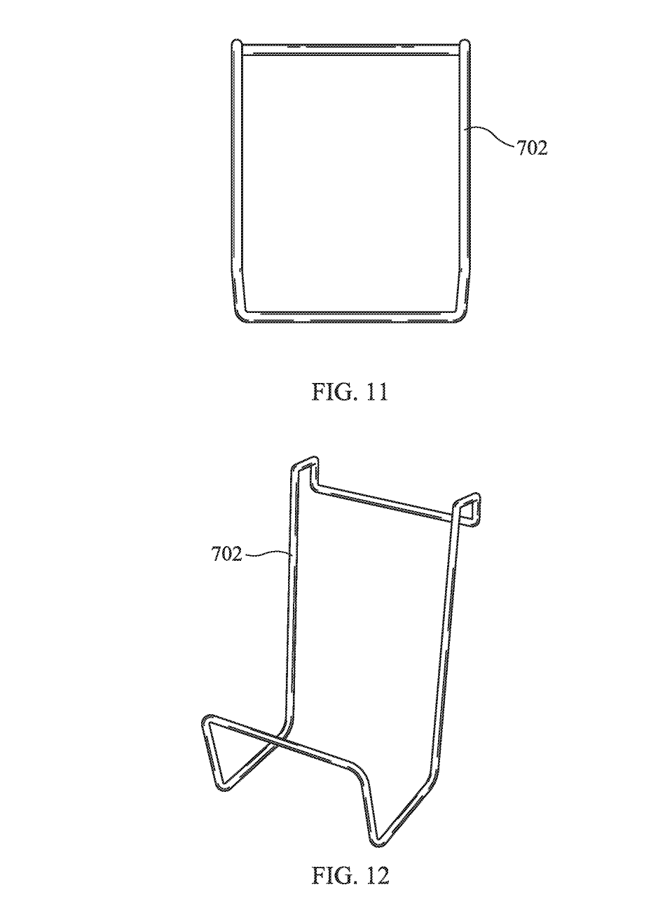

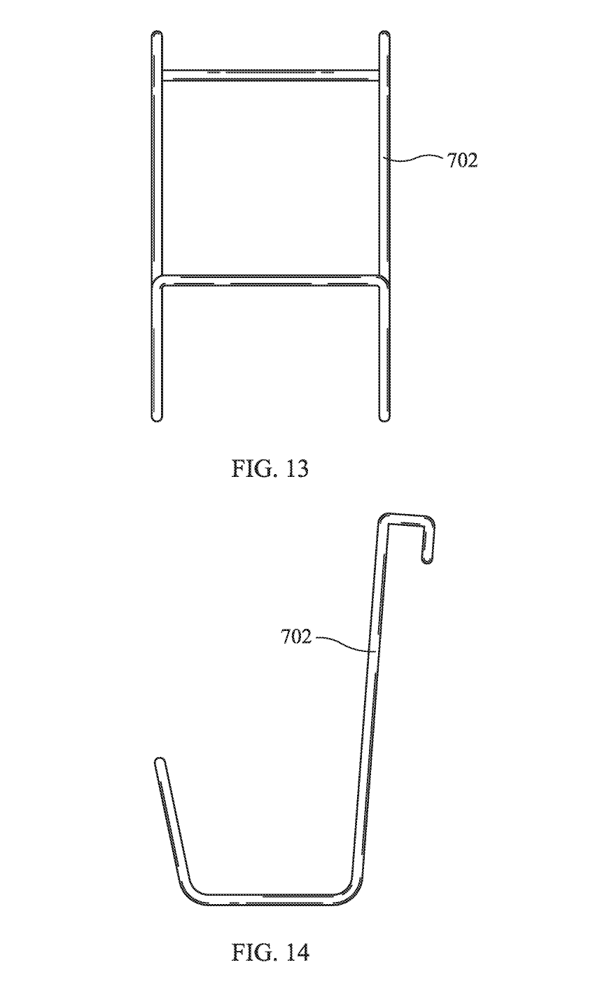

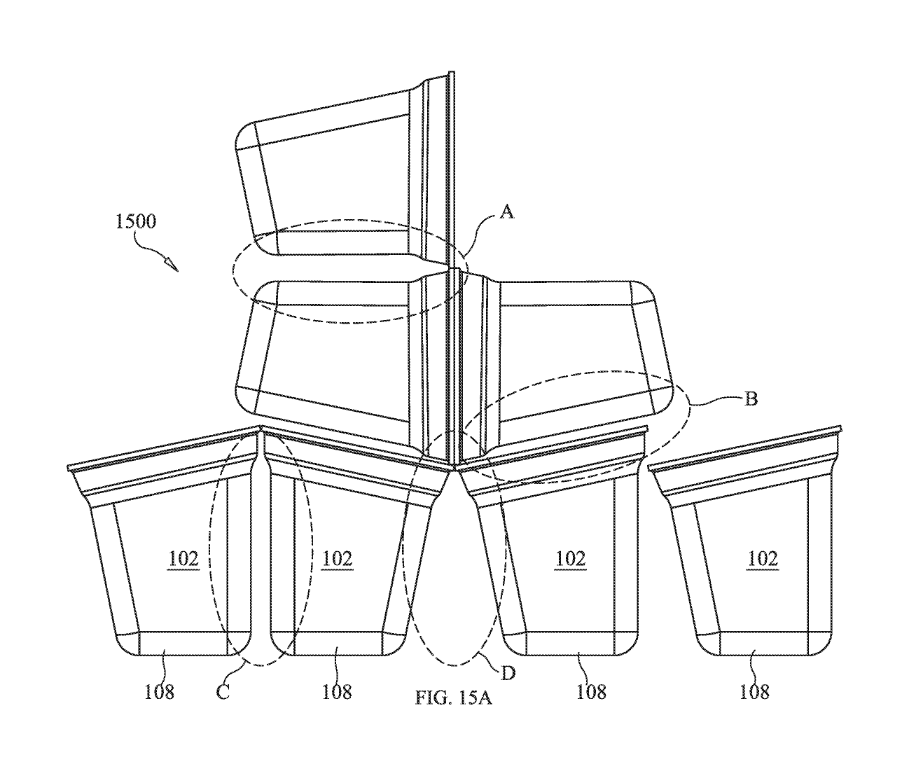

FIGS. 11 thru 14 illustrate various views of a metal wire-frame hanger 702 used as a mounting means for a disposable compost bin in an embodiment. FIG. 11 is a top view of the wire-frame hanger 702 for use in receiving and holding a disposable compost bin. In the illustrated embodiment, the width of the hanger 702 is generally in the range of 125 to 130 mm and the depth is generally in the range of 150 to 155 mm. FIG. 12 is a side perspective view of the wire-frame hanger 702. FIG. 13 is a front view of the wire-frame hanger 702 that depicts its height, which in the present embodiment is generally in the range of 210 to 215 mm. FIG. 14 is a side view of the wire-frame hanger 702 designed for receiving the disposable compost bin. Although a wire-frame hanger 702 is shown as one illustrative embodiment of a mounting means for use with the compost bin, other forms of retaining or mounting devices can be used to achieve similar objectives which are namely to hold the compost bin into position for routine receipt of disposed food waste or other organic matter. One alternative mounting means is a solid sheet metal hanger formed to have a structure that is generally similar in form factor to the wire-frame hanger 702.

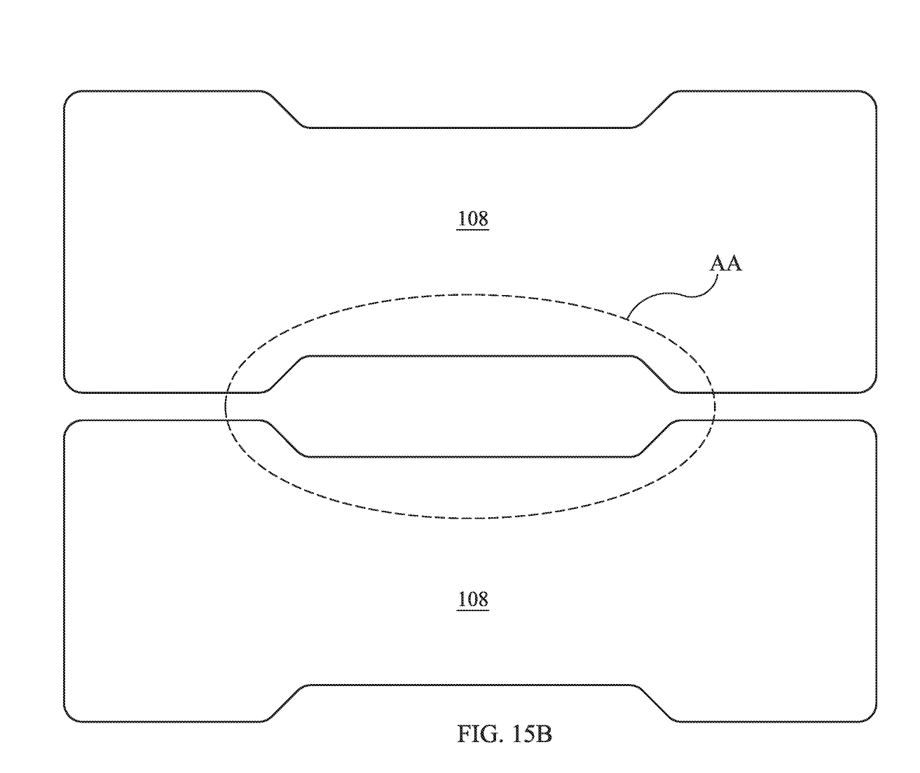

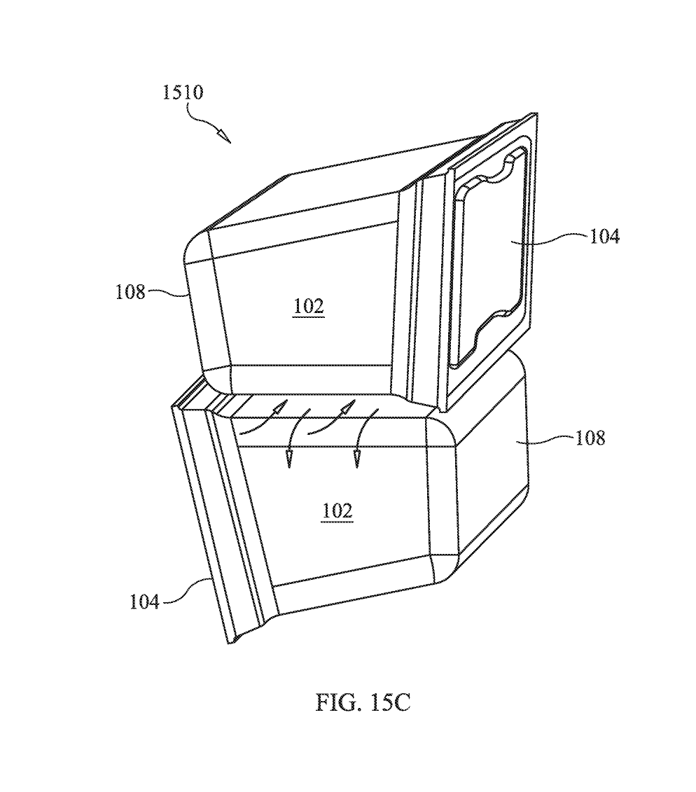

FIG. 15A depicts a representative collection of compost bins discarded in a compost heap or other composting collection resource. This collection 1500 shows several compost bins each having a container 102 and a closed bottom portion 108 and a plurality of open spaces between each wall-to-wall placement of the compost bins. As shown, the stacked wall-to-wall arrangement creates open air gaps between compost bins, which gaps are depicted as A, B, C and D. The air gaps A, B, C, D are areas where continuous air flow between compost bins occurs thereby enabling the aeration of the compostable materials in each compost bin. The trapezoidal shape of each compost bin combined with the compostable materials out of which each bin (i.e., containers 102 and lids 104) is made function together to provide optimal aeration for the compostable materials placed in each compost bin. In this manner, each compost bin is itself compostable and the composting process is enhanced by the flow of air through and between these structures. The composting process is further accelerated and improved with the addition of soil amendments that are either added by users to the refuse placed in each container 102 or integrated into the compostable materials during the manufacturing of each compost bin.

FIG. 15B depicts an air gap for air circulation between compost bins. As illustrated, the air gap AA is shown between two compost bins with containers having a generally trapezoidal design in a view looking upward from between the closed bottom portions 108 of the compost bins. The trapezoidal structure of both compost bins define an open gap AA when placed in an adjacent manner that permits the flow of air in a vertical direction between bins. This vertical flow combined with the previously described horizontal flow of air around each compost bin in a stacked wall-to-wall or even a side-to-side arrangement in a compost heap further improves the aeration of the compostable materials included in each compost bin.

FIG. 15C illustrates the general direction of air flow between two stacked compost bins in an embodiment. As illustrated, each bin has a container 102, a closed bottom portion 108, and a removable lid 104. This arrangement 1510 generally illustrates the flow of air passing between compost bins in a vertical orientation. Generally, compost bins are likely to be disposed of in a random, un-orderly manner in personal compost heaps or resources, communal compost collection resources, or in municipal compost collection sites and/or resources (e.g., separate collection bins used increasingly in dense metropolitan areas). However, where the compost bins are stacked upon each other or stacked side-to-side, this figure illustrates the general flow of air between the containers 102 that will tend to achieve optimal aeration for the enclosed compostable materials.

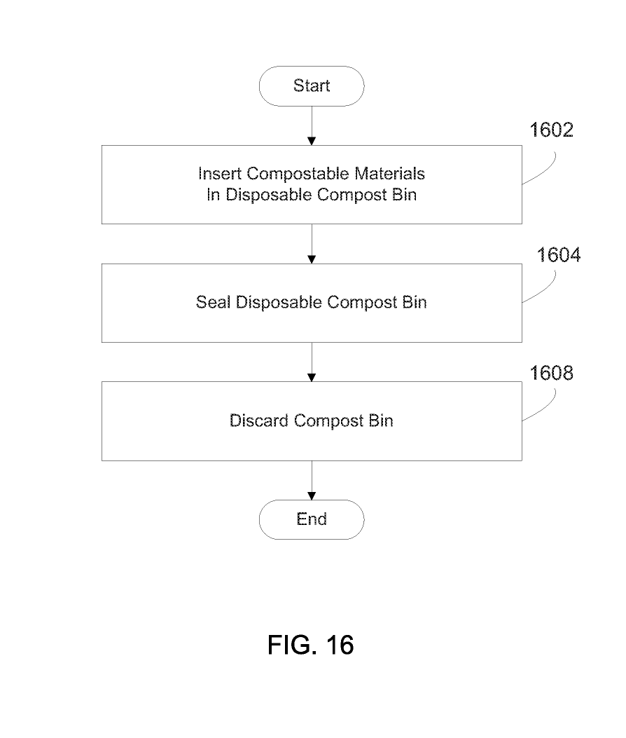

FIG. 16 is a flowchart illustrating one embodiment of a process for using a disposable compost bin. In the illustrated process, the user inserts compostable materials into a disposable compost bin, as shown at step 1602. The compostable materials include organic matter that can be disposed of or placed into the compost bin independently or with the addition of soil amendments such as coffee grounds, bone meal, blood meal, vermiculite, pumice or any combination of these materials. The compost bin itself is made of compostable plant-based fiber materials and is therefore also disposable into a compost heap. Among the compostable plant-based fiber materials of which the compost bin is comprised are wheat straw, wood fiber and sugarcane fiber. After compostable materials are placed into the compost bin, the compost bin is sealed using a removable lid, as shown at step 1604. The removable lid is also comprised of compostable materials such as wheat straw, wood fiber and sugarcane fiber. When the removable lid is placed onto a container of the compost bin it forms a vacuum-like seal with the container to enhance the aerobic composting process. Once sealed, a user discards the compost bin, as shown at step 1608, by placing it into a compost heap or other composting collection resource such as a backyard compost heap, a communal composting system or a municipal composting system.

As is generally known, an aerobic composting process requires significant aeration of compostable materials. Use of a disposable compost bin which is itself comprised of compostable materials greatly aids in the decomposition process. In addition, the shape of the compost bin further enhances the composting process since the shape is generally of either a trapezoidal design or a rectangular design and includes a plurality of panels and ridges for aerating the organic matter contained within the compost bin. The trapezoidal design is optimal because it enables the creation of air gaps between compost bins when they are placed in a compost heap or other collection resource. These air gaps enable a constant flow of air between compost bins in a stacked wall-to-wall arrangement or even in a side-to-side horizontal manner since each compost bin in this trapezoidal embodiment includes a shaped groove area that defines an air space or air gap between co-located compost bins in a compost heap. Alternative embodiments of the containers 102 used in each compost bin also enable some level of air flow and improved composting though the preferred embodiment is one that uses containers 102 with trapezoidal designs.

Although specific embodiments have been illustrated and described herein, it will be appreciated by those of ordinary skill in the art that a wide variety of alternate and/or equivalent implementations may be substituted for the specific embodiments shown and described without departing from the scope of the present disclosure. This application is intended to cover any adaptations or variations of the embodiments discussed herein. It is to be understood that any particular embodiment shown and described by way of illustration is in no way intended to be limiting and references to details of various embodiments are not intended to limit the scope of the claims presented below.

What is claimed is:

1. A disposable compost bin for receiving compostable materials, the bin comprising:
a container comprised of a compostable material, the container having a closed bottom portion, a continuous wall defining an open upper portion, and a continuous lid receiving edge on the open upper portion, the container having a continuous inner surface and a shaped groove on a first outer surface;
a removable lid having a substantially rectangular shape and a continuous outer edge, the lid insertably placed on the continuous lid receiving edge of the container for sealing the received compostable materials, the removable lid forming a vacuum-like seal when insertably placed on the continuous lid receiving edge; and
a mounting means received in the shaped groove for holding the container, the mounting means securing the container to a side wall structure.

2. The compost bin of claim 1 wherein the compostable material of the container is a plant-based fiber material, the plant-based fiber material being at least one of a wheat straw material, a wood fiber material, and a sugar cane fiber material.

3. The compost bin of claim 1 wherein the removable lid is comprised of a plant-based fiber material, the plant-based fiber material being at least one of a wheat straw material, a wood fiber material, and a sugar cane fiber material.

4. The compost bin of claim 1 wherein the continuous wall forms at least one of a trapezoidal design and a rectangular design, each design extending from the closed bottom portion to the defined open upper portion.

5. The compost bin of claim 4 wherein the trapezoidal design enables aeration of the received compostable materials in each of a plurality of containers when the plurality of containers are in a wall-to-wall stacked arrangement in a compost heap.

6. The compost bin of claim 1 wherein the container further comprises an emulsified wax additive.

7. The compost bin of claim 6 wherein the emulsified wax additive is Alkyl Ketene Dimer (AKD).

8. The compost bin of claim 1 wherein the mounting means is a metal wire-frame hanger, the metal wire-frame hanger having a first end received in the shaped groove and a second end secured to the side wall structure.

9. The compost bin of claim 1 wherein the mounting means is a solid metal hanger, the solid metal hanger having a first end received in the shaped groove and a second end secured to the side wall structure.

10. The compost bin of claim 1 wherein the side wall structure is a cabinet door.

11. A disposable compost bin for receiving compostable materials, the bin comprising:
a container comprised of a compostable material, the container having a closed bottom portion, a continuous wall defining an open upper portion, and a continuous lid receiving edge on the open upper portion, the container having a continuous inner surface; and
a removable lid having a substantially rectangular shape and a continuous outer edge, the lid insertably placed on the continuous lid receiving edge of the container for sealing the received compostable materials, the removable lid forming a vacuum-like seal when insertably placed on the continuous lid receiving edge.

12. The compost bin of claim 11 wherein the compostable material of the container is a plant-based fiber material, the plant-based fiber material being at least one of a wheat straw material, a wood fiber material, and a sugar cane fiber material.

13. The compost bin of claim 11 wherein the removable lid is comprised of a plant-based fiber material, the plant-based fiber material being at least one of a wheat straw material, a wood fiber material, and a sugar cane fiber material.

14. The compost bin of claim 11 wherein the continuous wall forms at least one of a trapezoidal design and a rectangular design, each design extending from the closed bottom portion to the defined open upper portion.

15. The compost bin of claim 14 wherein the trapezoidal design enables aeration of the received compostable materials in each of a plurality of containers when the plurality of containers are in a wall-to-wall stacked arrangement in a compost heap.

16. The compost bin of claim 11 wherein the container further comprises an emulsified wax additive.

17. The compost bin of claim 16 wherein the emulsified wax additive is Alkyl Ketene Dimer (AKD).

18. The compost bin of claim 11 wherein the side wall structure is a cabinet door.

19. A method of composting organic matter, the method comprising:
inserting the organic matter into a container, the container comprised of a compostable material, the container having a closed bottom portion, a continuous wall defining an open upper portion, and a continuous lid receiving edge on the open upper portion, the container having a continuous inner surface;
sealing the inserted organic matter in the container using a removable lid, the removable lid having a substantially rectangular shape and a continuous outer edge, the lid insertably placed on the continuous lid receiving edge of the container for forming a vacuum-like seal with the container; and
discarding the sealed container into a composting collection resource.

20. The method of claim 19 wherein the inserting of the organic matter further comprises inserting the contents of one or more packets of soil amendments into the organic matter, the soil amendments comprising at least one of coffee grounds, bone meal, blood meal, vermiculite and pumice.

21. The method of claim 19 wherein the inserting of the organic matter uses a container having a trapezoidal design, the trapezoidal design including a plurality of panels and ridges for aerating the organic matter.

22. The method of claim 19 wherein the composting collection resource is one of a group consisting of a personal composting system, a communal composting system and a municipal composting system.

23. The method of claim 19 wherein the compostable material is comprised of a plant-based fiber material, the plant-based fiber material being at least one of a wheat straw material, a wood fiber material, and a sugar cane fiber material.

24. The method of claim 23 wherein the removable lid is comprised of the compostable material.
